(19) United States
(12) Patent Application Publication
(10) Pub. No.: US 2024/0273436 A1
(43) Pub. Date: Aug. 15, 2024

(54) TIME TRACKING DEVICE AND LABORATORY SYSTEM

(71) Applicant: Roche Diagnostics Operations, Inc., Indianapolis, IN (US)

(72) Inventors: Thomas Heinemeyer, Penzberg (DE); Dieter Kempken, Penzberg (DE); Marian Roetzer, Penzberg (DE); Lorenz Scheinpflug, Penzberg (DE); Florian Schweinberger, Penzberg (DE)

(21) Appl. No.: 18/568,254

(22) PCT Filed: Jun. 8, 2022
(86) PCT No.: PCT/EP2022/065474

§ 371 Date: Dec. 7, 2023

(30) Foreign Application Priority Data
Jun. 9, 2022 (EP) 21178451.7

(51) Int. Cl. G06Q 10/0633; G04F 1/00

(52) U.S. Cl. CPC G06Q 10/0633; G04F 1/005

(57) ABSTRACT

A time tracking device (112) for monitoring system performance of at least one laboratory instrument is proposed. The time tracking device (112) comprises at least one support structure (114). The time tracking device (112) further comprises at least one electronics unit (116) and at least one motion sensor (118) housed by the support structure (114). The motion sensor (118) is configured for detecting a change of motion and/or orientation of the time tracking device (112). The electronics unit (116) is configured for measuring time, wherein a time measurement is initiated by detecting a change of motion and/or orientation of the time tracking device (112) and is terminated by detecting a subsequent change of motion and/or orientation of the time tracking device (112). The support structure (114) is a polygonal support structure (114) comprising a plurality of faces (120), wherein the support structure (114) comprises at least two interactive faces (136). Each of the interactive faces (136) comprises at least one user interface (138) comprising at least one display device (140). Each of the interactive faces (136) is configured for displaying at least one status indication (171) of the laboratory instrument, wherein the interactive face (136) matching a current status of the laboratory instrument is selectable by a user via changing motion and/or orientation of the time tracking device (112). The time tracking device (112) is con-figured for providing data relating to the measured time via at least one communication interface (172).

### TECHNICAL FIELD

The invention refers to a time tracking device for monitoring system performance of at least one laboratory instrument and a laboratory system. The devices according to the present invention may mainly be used by laboratory staff to support and facilitate workflows. Other applications are generally feasible.

### BACKGROUND ART

Planning of resources such as of machinery and personnel in laboratories comprising laboratory instruments is usually performed by estimating system performance instead of hard facts and numbers and, in particular not in real time. This may lead to unnecessary instrument downtime and unused resources and in consequence to unnecessary costs. Known time tracking machinery for monitoring system performance in laboratories are usually not profitable. Information sharing of instrument status and other instrument related information is often a cumbersome incubation.

WO 2009/140967 A1 describes a computer system for analyzing a specimen processing workflow in a pathology laboratory. The computer system includes a display and a processor configured to provide a first interface, a second interface, and a third interface to the display. The processor is further configured to receive, via the first interface, a first set of parameters associated with an existing specimen processing workflow in a pathology laboratory. The first set of parameters includes a workflow process and a workflow scale. The processor is further configured to determine, based on the first set of parameters, current performance data of the existing specimen processing workflow having an associated current cost information and current time utilization.

WO 2018/081379 A1 describes a method for remotely monitoring performance of experimental studies associated with laboratory instruments. The method comprises for a subject laboratory instrument: a) transmitting setup data to a location of the subject laboratory instrument on which a requested study comprising one or more tests is to be performed; b) updating a set of one or more study progress values for the subject laboratory instrument indicating the requested study is either complete or in progress; and c) updating a set of one or more data collection progress values for the subject laboratory instrument indicating data for the requested study has been captured or reviewed or printed.

US 2020/319602 A1 describes a timer apparatus. A shape of the timer apparatus includes a cube, a pyramid, a pentagonal prism, a hexagonal prism, or a polygonal prism. The shape of the timer apparatus is preferable a cube. The timer apparatus also includes timing surface(s). Each of the timing surface(s) displays a value in minutes/seconds from which to countdown. The timer apparatus also includes a display surface. The display surface provides a timer and controls associated with the timer apparatus. The timer is configured to countdown from the value displayed by the timing surface when the timing surface is detected in a top position. The timer apparatus also includes a stop surface. When the stop surface is detected in the top position, the timer is reset. Furthermore, the timer apparatus includes a controller and an orientation sensor.

### Problem to be Solved

It is therefore an objective of the present invention to provide a time tracking device for monitoring system performance of at least one laboratory instrument and at least one laboratory system which at least partially avoid the shortcomings of known devices and methods of this kind and which at least partially address the abovementioned challenges. Specifically, devices and methods shall be disclosed which allow simplified and precise time tracking measurements for monitoring system performance of at least one laboratory instrument.

### SUMMARY

This problem is addressed by a time tracking device for monitoring system performance of at least one laboratory instrument and at least one laboratory system with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a time tracking device for monitoring system performance of at least one laboratory instrument is disclosed.

The term “laboratory” as used herein is a broad term and is to be given its ordinary and customary meaning to a person of ordinary skill in the art and is not to be limited to a special or customized meaning. The term specifically may refer, without limitation, to at least one environment comprising at least one laboratory instrument. The laboratory may be a location configured for work in the field of the natural sciences and/or engineering in the sense that it offers the opportunity to conduct corresponding measurements and controls.

The laboratory may be designed as in-vitro diagnostics laboratory. The term “in-vitro diagnostics” as used herein is a broad term and is to be given its ordinary and customary meaning to a person of ordinary skill in the art and is not to be limited to a special or customized meaning. Specifically, in-vitro diagnostics may comprise performing at least one test on a sample, e.g. a biological sample that has been taken off the human body or animal body. For example, the test on the sample may comprise applying at least one reagent to the sample and monitoring a detectable reaction. For example, the laboratory may be a clinical laboratory, a medical laboratory, a forensic laboratory or a blood bank.

The term “biological sample” as used herein is a broad term and is to be given its ordinary and customary meaning to a person of ordinary skill in the art and is not to be limited to a special or customized meaning. The term specifically may refer, without limitation, to at least one biological material that may potentially comprise at least one analyte of interest. For example, the biological sample may comprise a body fluid, such as blood, interstitial fluid, urine, saliva or other types of body fluids. For example, the biological sample may be or may comprise an aliquot of a substance such as a biological compound. Specifically, the biological sample may be or may comprise at least one biological specimen, such as one or more of: blood; blood serum; blood plasma; urine; saliva. The biological sample may be a liquid sample. For example, the liquid sample may be or may comprise at least one pure liquid, such as a liquid substance and/or a solution containing one or more liquid substances, comprising the biological substance. The liquid sample may be or may comprise a liquid mixture, such as a suspension, an emulsion and/or a dispersion of biological substances. The biological sample may be a solid sample, for example, at least one sample of tissue.

The term “laboratory instrument” as used herein is a broad term and is to be given its ordinary and customary meaning to a person of ordinary skill in the art and is not to be limited to a special or customized meaning. The term specifically may refer, without limitation, to an arbitrary device of the laboratory configured for performing at least one function such as analysis of the biological sample. The laboratory instrument may be one or more of a pre-analytic laboratory instrument, an analytic laboratory instrument, or a post-analytic laboratory instrument.

The term “time tracking” as used herein is a broad term and is to be given its ordinary and customary meaning to a person of ordinary skill in the art and is not to be limited to a special or customized meaning. The term specifically may refer, without limitation, to a process of measuring a time duration of a status of the laboratory instrument. The term “time tracking device” as used herein is a broad term and is to be given its ordinary and customary meaning to a person of ordinary skill in the art and is not to be limited to a special or customized meaning. The term specifically may refer, without limitation, to a device configured for performing time tracking. The time tracking device may comprise at least one timer or clock for measuring time. The timer or clock may be electronically driven. The time tracking device, additionally or alternatively, may be designed for receiving an external time signal such as from an atomic clock via a satellite television or computer connection. The time tracking device may be configured for measuring time with a precision in ms or μs range.

The term “system performance” as used herein is a broad term and is to be given its ordinary and customary meaning to a person of ordinary skill in the art and is not to be limited to a special or customized meaning. The term specifically may refer, without limitation, to information about times of use, information about availability, information about system downtime such as information about repair times, information about maintenance times, information about times the laboratory instrument is out of order, information about times the laboratory instrument is offline. The term “monitoring system performance” as used herein is a broad term and is to be given its ordinary and customary meaning to a person of ordinary skill in the art and is not to be limited to a special or customized meaning. The term specifically may refer, without limitation, to determining system performance of the laboratory instrument at at least two different time points such as over a period of time. The monitoring may comprise one or more of documenting the system performance, determining at least one time log, preparing at least one report about the system performance such as a manual report.

The time tracking device comprises at least one support structure. The time tracking device comprises at least one electronics unit and at least one motion sensor housed by the support structure. The motion sensor is configured for detecting a change of motion and/or orientation of the time tracking device. The electronics unit is configured for measuring time, wherein a time measurement is initiated by detecting a change of motion and/or orientation of the time tracking device and is terminated by detecting a subsequent change of motion and/or orientation of the time tracking device. The support structure is a polygonal support structure comprising a plurality of faces. The support structure comprises at least two interactive faces. Each of the interactive faces comprises at least one user interface comprising at least one display device. Each of the interactive faces is configured for displaying at least one status indication of the laboratory instrument. The interactive face matching a current status of the laboratory instrument is selectable by a user via changing motion and/or orientation of the time tracking device. The time tracking device is configured for providing data relating to the measured time via at least one communication interface.

The time tracking device comprises the at least one support structure. The term “support structure” as used herein is a broad term and is to be given its ordinary and customary meaning to a person of ordinary skill in the art and is not to be limited to a special or customized meaning. The term specifically may refer, without limitation, to an arbitrary shaped three-dimensional body. The support structure may be an element which is configured for fully or partially enclosing and/or covering one or more components and for providing protection for these one or more components, such as against environmental influences and/or mechanical influence and/or humidity. The support structure may be configured as mounting base and/or housing. The support structure, specifically, may be a rigid support structure made of one or more of a plastic material, a metallic material or a cardboard material. For example, the support structure may comprise at least one polygonal solid shell, for example a plastic shell or metal shell. The term “at least partially enclosing” as used herein is a broad term and is to be given its ordinary and customary meaning to a person of ordinary skill in the art and is not to be limited to a special or customized meaning. The term specifically may refer, without limitation, to embodiments in which the support structure completely encloses the named components of the time tracking device and to embodiments in which parts or elements of the time tracking device are uncovered by the support structure. For example, the support structure may comprise cutouts and/or windows in which the user interface may be insertable such that screens of the display devices are visible from outside of the time tracking device.

The time tracking device comprises the at least one electronics unit. The term “electronics unit” as generally used herein is a broad term and is to be given its ordinary and customary meaning to a person of ordinary skill in the art and is not to be limited to a special or customized meaning. The term specifically may refer, without limitation, to at least one unit of the time tracking device configured for one or more of performing the time measurement, storing data, processing data, providing power to further components of the time tracking device and the like. The electronics unit may comprise at least one processor, such as at least one microprocessor. The processor may be an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric coprocessor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor such as a Raspberry Pi® or Arduino® based microprocessor. Specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) or the like. The processor specifically may be configured, such as by software programming, for performing one or more evaluation operations.

The time tracking device comprises the at least one motion sensor configured for detecting a change of motion and/or orientation of the time tracking device. The term “motion sensor” as used herein is a broad term and is to be given its ordinary and customary meaning to a person of ordinary skill in the art and is not to be limited to a special or customized meaning. The term specifically may refer, without limitation, to a sensor device having at least one sensor element configured for detecting a change of motion and/or orientation of the time tracking device. The change of motion may be an acceleration along at least one axis such as in a horizontal plane, in a vertical plane and/or around an axis of rotation. For example, the change of motion may comprise a shaking of the time tracking device. The change of orientation may comprise at least one movement of the time tracking device around an axis of rotation. The change of orientation may comprise for example rotating and/or flipping and/or scrolling of the time tracking device. For example, the motion sensor may comprise at least one gyrosensor.

The electronics unit is configured for measuring time. The electronics unit may comprise at least one timer or clock for measuring time. The timer or clock may be electronically driven. The electronics unit, additionally or alternatively, may be designed for receiving an external time signal such as from an atomic clock via a satellite television or computer connection. The electronics unit may control the time measurement. A start of the time measurement may be initiated by the motion sensor detecting a change of motion and/or orientation of the time tracking device. The motion sensor may be configured for providing a sensor signal to the electronics unit. The electronics unit may be configured upon receiving the sensor signal to start the time measurement. The time measurement may be terminated by the motion sensor detecting a subsequent change of motion and/or orientation of the time tracking device and providing a further sensor signal to the electronics unit. The electronics unit may be configured upon receiving the sensor signal to end the time measurement.

The electronics unit may comprise at least one memory configured for storing a time log. The term “memory” as used herein is a broad term and is to be given its ordinary and customary meaning to a person of ordinary skill in the art and is not to be limited to a special or customized meaning. The term specifically may refer, without limitation, to a storage medium may refer to a non-transitory data storage means, such as a hardware storage medium having stored thereon computer-executable instructions. The memory may be or may comprise a storage medium such as a random-access memory (RAM) and/or a read-only memory (ROM). The term “time log” as used herein is a broad term and is to be given its ordinary and customary meaning to a person of ordinary skill in the art and is not to be limited to a special or customized meaning. The term specifically may refer, without limitation, to a protocol comprising time-resolved recording of events, in particular relating to status changes of the laboratory instrument.

The support structure is a polygonal support structure comprising a plurality of faces. The term “face” as used herein is a broad term and is to be given its ordinary and customary meaning to a person of ordinary skill in the art and is not to be limited to a special or customized meaning. The term specifically may refer, without limitation, to exterior surfaces of the support structure. The support structure may be a three dimensional structure selected from the group consisting of: a pyramid; a lying trigonal prism, a lying pentagonal prism, a lying hexagonal prism, a cube. The time tracking device may be a mobile and/or portable time tracking device. The dimensions of the time tracking device may be adapted to a diameter of a hand of an average adult. For example, a diameter of the time tracking device may be in a range from 5 to 10 cm.

The support structure comprises at least two interactive faces. The term “interactive face” as used herein is a broad term and is to be given its ordinary and customary meaning to a person of ordinary skill in the art and is not to be limited to a special or customized meaning. The term specifically may refer, without limitation, to a face of the support structure configured for providing a uni- or bi-directional interface for a user of the time tracking device. Each of the interactive faces comprises at least one user interface comprising at least one display device. The term “user interface” as used herein is a broad term and is to be given its ordinary and customary meaning to a person of ordinary skill in the art and is not to be limited to a special or customized meaning. The term may refer, without limitation, to a feature of the time tracking device which is configured for interacting with its environment, such as for the purpose of unidirectionally or bidirectionally exchanging information, such as for exchange of one or more of data or commands. For example, the user interface may be a bi-directional interface. The user interface may be configured to share information with a user and to receive information by the user. The user interface may be configured for visually interacting with a user. Therefore, the user interface comprises the at least one display device.

The term “display device” is a broad term and is to be given its ordinary and customary meaning to a person of ordinary skill in the art and is not to be limited to a special or customized meaning. The term specifically may refer, without limitation, to at least one electronic device comprising at least one display. As further used herein, the term “display” is a broad term and is to be given its ordinary and customary meaning to a person of ordinary skill in the art and is not to be limited to a special or customized meaning. The term specifically may refer, without limitation, to an arbitrary shaped device configured for displaying an item of information such as at least one image, at least one diagram, at least one histogram, at least one text, at least one sign. Specifically, the display device comprises the at least one screen. The screen may have an arbitrary shape such as a rectangular shape. The shape of the screen may be adapted to the shape of the support structure. The display device may comprise one or more of a liquid crystal display (LCD), a thin-film transistor (TFT) display, a light emitting diode (LED) display, an organic light emitting diode (OLED) display, in particular an OLED foil display, an eINK® display. The display device may be touch sensitive and/or gesture sensitive. For example, the display device may be configured such that simple gestures trigger one or more actions of the time tracking device, e.g. shaking to start time measurement, tapping twice to go back to a home screen, and the like. The time tracking device may comprise electrical connections between the display device and the electronics unit, e.g. for controlling the display device via the electronics unit, providing power to the display device and the like.

Each of the interactive faces, via the display device, may be configured for displaying at least one status indication of the laboratory instrument. The status of the laboratory instrument may be one or more of in use, standby, maintenance, repair, out of order, offline. The term “status indication” is a broad term and is to be given its ordinary and customary meaning to a person of ordinary skill in the art and is not to be limited to a special or customized meaning. The term specifically may refer, without limitation, to an arbitrary visual indication. The visual indication may comprise using light of a specific wavelength indicating a status of the laboratory instrument, text and/or numbers and/or characters indicating a status of the laboratory instrument.

The status indication of the laboratory instrument displayed by the respective interactive face may be assignable to the respective interactive face. The electronics unit may be configured for controlling, e.g. setting, the status indication displayed by the interactive faces. The electronics unit may be controllable by a remote control, such as via at least one computer connected with the time tracking device, such that the status indication may be assignable by the remote control. For example, the status indication may be assigned depending on the type of laboratory instrument and/or history of laboratory instrument.

Each of the interactive faces may be configured for displaying one status indication of the laboratory instrument irrespectively of a number of turns of the time tracking device. For example, the time tracking device may allow selecting six different status indications, wherein the support structure comprises 6 interactive faces each for one status indication.

The time tracking device may be configured for hyperscrolling. The status indication displayed by the interactive faces may depend on a number of turns of the time tracking device. For example, the time tracking device may allow selecting 6+x different status indications. The support structure may comprise 6 interactive faces, wherein the status indications become visible through overtwisting the time tracking device. The time tracking device may be configured as virtual scroll wheel having more or less pages than sides on the support structure.

The term “current status” is a broad term and is to be given its ordinary and customary meaning to a person of ordinary skill in the art and is not to be limited to a special or customized meaning. The term specifically may refer, without limitation, to an actual status of the laboratory instrument. The interactive face matching a current status of the laboratory instrument is selectable by a user via changing motion and/or orientation of the time tracking device. For example, the time tracking device may comprise at least one selection position. The time tracking device may be configured for selecting a current status of the laboratory instrument by bringing the interactive face corresponding the current status of the laboratory instrument into the selection position. The selection position may be defined with respect to direction of the gravitational force. The selection position may be a position in which the interactive face to be selected is oriented upwards, i.e. the direction of the gravitational force being a normal of a plane of the interactive face. However, other selection positions may be possible. The selection position may be defined depending on the three dimensional structure of the support structure. For example, in case of a cube the selection position may be a position in which the interactive face to be selected is oriented upwards. For example, in case of a lying hexagon, the selection position may be a position in which the interactive face to be selected forms an angle of 30° with the direction of the gravitational force. The selection position may be defined depending on a surface the time tracking device is arranged on such as a surface of a charging element. The charging element may also be referred to as a charging station. The orientation with respect to the direction of the gravitational force may be determined by the motion sensor such as the gyrosensor. Additionally or alternatively, the status indication matching to a current status of the laboratory instrument may be selected by using at least one gesture such as tapping on an interactive face.

The electronics unit may be configured for assigning the measured time to a status of the laboratory instrument depending on the selected interactive face.

The electronics unit may be configured for controlling the display devices. As outlined above, the time tracking device may be moved such as turned or flipped. Specifically, the controlling may comprise adapting an orientation of information displayed by the display devices depending on orientation of the time tracking device. This may allow user friendly reading of the status indication by a user.

The interactive face may be configured for showing additional information. The interactive face may be configured for displaying one or more of at least one warning message, at least one maintenance information, information about an owner of the laboratory instrument, information about at least one further laboratory instrument, a menu, at least one readout of at least one sensor, messages from third party applications and/or sensors, data sources in general, previous status information, details from an instrument related documentation system such as Digital Instrument Book, booking status of the instrument feed by an external database such as booking system and/or activity planner. For example, the interactive face may be configured for displaying cross information from further time tracking devices and/or laboratory instruments of the laboratory, e.g. for showing errors of other analyzers in near proximity. This may allow not showing only a limited number of information to a user but information relating to usage of the laboratory instrument.

Each of the faces of the support structure may comprise at least one light emitter configured for emitting light in a plurality of wavelengths. The light emitter may be at least one light emitting diode (LED). For example, in case of the interactive faces the light emitter may be the display device and/or at least one further light emitter such as an LED. A wavelength may be assigned to each of the status indications, wherein the electronics unit may be configured for switching the wavelengths of the light emitters depending on the interactive face selected by the user.

The time tracking device may comprise at least one sensor configured for monitoring environmental conditions. The sensor may be selected from the group consisting of: at least one temperature sensor, at least one pressure sensor; at least one humidity sensor; at least one sensor configured for detecting magnetic interferences; at least one location sensor such as at least one GPS sensor, Glonass sensor, Galileo sensor, at least one Bluetooth low energy (BLE) sensor. The at least one sensor may monitor the environmental conditions such as temperature, pressure, humidity. Those measures may help to troubleshoot failure of instrument errors. Those measures may also add additional layers of information to measurement data of the laboratory instrument. The GPS sensor may be configured for automatically assigning instrument location. This may be particularly useful for multiple locations or buildings.

The time tracking device may comprise at least one reader such as at least one RFID reader and/or at least one NFC (near field communication) reader. The reader may be configured for receiving information about a user from at least one identifier, such as an tag, card or the like, of the user. For example, the RFID reader may be configured for receiving information about a user from at least one RFID identifier, such as an RFID tag, of the user. The term “RFID identifier” as used herein is a broad term and is to be given its ordinary and customary meaning to a person of ordinary skill in the art and is not to be limited to a special or customized meaning. The term specifically may refer, without limitation, to a label configured for exchanging data information with the RFID reader by using radiofrequency electromagnetic radiation, in particular by using the NFC standard. The RFID identifier may further comprise an antenna configured to receive and to emit a radiofrequency signal and an electronic chip such as a microchip configured to store the data information. Specifically, the RFID identifier may be a flexible substrate having an electronic conducting coil and optionally at least one microchip. The electronics unit may be configured for combining the measured time with the information about the user. Adding an RFID reader to combine system usage with the user may allow having the potential to assess productivity and/or reveal user's best practice. For example, the RFID identifier may be realized using available infrastructure to identify the user e.g. via company ID. For example, the time tracking device may comprise at least one NFC chip and a NFC reader may be used. Using RFID may allow communication over larger distances compared to NFC.

The time tracking device may comprise at least one fingerprint sensor. The fingerprint sensor may be configured for receiving information about a user from reading at least one finger print from the user. Fingerprint sensors are known by the skilled person. The fingerprint sensor may be used in combination with an access control system to control the access to the time tracking device and the connected laboratory instrument. The electronics unit may be configured for combining the readout of the fingerprint via the fingerprint sensor and a stored at least one fingerprint of the user in an access control system to control the access to the laboratory instrument or further elements of the laboratory. For example, a fingerprint identifier may be realized using available infrastructure to identify the user via his fingerprint.

The time tracking device may comprise at least one electronic camera. Suitable electronic cameras are known by the skilled person.

The electronic camera may be configured to read barcodes such as 1D barcodes, 2D barcodes or QR codes.

The electronic camera may be further configured to capture images. These images may help to troubleshoot failure of instrument errors. The images may add additional layers of information to measurement data of the laboratory instrument. The images may be used e.g. for system failure documentation when those images are stored in a database or electronic laboratory book or those images are send via electronic communication systems to the system support.

The electronic camera may be further configured for face recognition. The electronic camera may be configured for receiving an images of the face of a user. An electronic camera may be used in combination with an access control system to control the access to the time tracking device and the connected laboratory instrument. The electronics unit may be configured for combining the readout of the electronic camera (an image) and the stored at least one face image of the user in an access control system to control the access to the system. For example, the electronic camera may be realized using available infrastructure to identify the user via his face image.

The time tracking device may comprise at least one rechargeable energy unit. The rechargeable energy unit may comprise at least one rechargeable battery. The rechargeable energy unit may be part of the electronics unit or may be a separate unit. For example, the rechargeable energy unit may comprise at least one battery housing configured for receiving at least one rechargeable battery. The rechargeable energy unit may be configured for wireless charging. The time tracking device may comprise at least one connector for charging the rechargeable energy unit. The connector may be arranged at a face of the support structure. For example, the time tracking device may comprise at least one qi charger, wherein the qi charger is placed on a charging station for charging the rechargeable energy unit.

The time tracking device may comprise at least one speaker unit configured for providing audible information. The time tracking device may comprise at least one microphone unit configured for receiving voice commands. Speaker unit and microphone may allow using the time tracking device as laboratory journal such as for dictate and documentation. This may allow “Hands free documentation”. The time tracking device may be designed for hands-free digital data capture, for example by using voice recognition. The time tracking device may be designed for data capture at the laboratory instrument assigned and/or connected to the time tracking device. The time tracking device may be designed for integrating data from other sources such as external sources and/or from previous time points. The time tracking device may be part of a communication network enabling exchange with other devices, for example informatics system.

The time tracking device may be configured for providing data relating to the measured time via the at least one communication interface. The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. In particular, the communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may specifically provide means for transferring or exchanging information. In particular, the communication interface may provide a data transfer connection, e.g. Bluetooth, NFC, inductive coupling or the like. As an example, the communication interface may be or may comprise at least one port comprising one or more of a network or internet port, a USB-port and a disk drive. The communication interface may be at least one web interface. The time tracking device may be remote controllable by receiving commands via the communication interface.

The time tracking device may comprise at least one switch, specifically at least one micro switch. The electronic unit may be configured for performing at least one predefined action triggered by activating the switch. The predefined action comprises one or more of providing information that the laboratory instrument breaks down to a further device via the communication interface, switching off the time tracking device, put the time tracking device into standby, or providing an automated message to a next user and/or further device via the communication interface that the laboratory instrument is available. Integration of switches may allow triggering predefined actions in the background, e.g. instrument breaks down, cube is turned to out of order, service is alarmed, second idea: early switch to ‘standby’ when compared to booking calendar, automated message to next user or service in line that systems is available earlier.

In a further aspect of the present invention, a laboratory system is disclosed.

The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components parts forming a whole. Specifically, the components may interact with each other in order to fulfill at least one common function.

The at least two components may be handled independently or may be coupled or connectable. The term “laboratory system” as used herein is a broad term and is to be given its ordinary and customary meaning to a person of ordinary skill in the art and is not to be limited to a special or customized meaning. The term specifically may refer, without limitation, to a group of at least two elements or components which are capable of interacting in a laboratory.

The laboratory system comprising at least one laboratory instrument and at least one time tracking device according to the present invention. The laboratory system further comprises at least one processing device. The processing device comprises at least one communication interface configured for receiving data relating to the measured time from the communication interface of the time tracking device. The processing device comprises at least one evaluation device configured for monitoring system performance of the laboratory instrument by evaluating the data relating to the measured time received from the communication interface of the time tracking device.

The term “processing device” as used herein is a broad term and is to be given its ordinary and customary meaning to a person of ordinary skill in the art and is not to be limited to a special or customized meaning. The term specifically may refer, without limitation, to an arbitrary electronic circuit configured for operating on data. In particular, the processing device may be configured for performing operations on data received via the communication interface. As an example, the processing device may specifically be or may comprise an integrated circuit (IC), such as an application-specific integrated circuit (ASIC) or a field programmable gate array (FPGA).

The term “evaluation device” as used herein is a broad term and is to be given its ordinary and customary meaning to a person of ordinary skill in the art and is not to be limited to a special or customized meaning. The term specifically may refer, without limitation, to

The evaluation device may be configured for predicting instrument usage and/or downtime by evaluating the data relating to the measured time received from the communication interface of the time tracking device. The evaluating of the data relating to the measured time received from the communication interface of the time tracking device may comprise using an algorithm for predicting instrument usage and/or downtime. For example, the evaluation device may be configured for determining one or more of when is an ideal point for maintenance, which time and day of the week is best as less interfering with usage, what are future realistic equipment usage times based on obtained data and the like.

The laboratory system may comprise a plurality of laboratory instruments. The laboratory system may comprise a plurality of time tracking devices each assigned to one of the laboratory instruments. The laboratory system may comprises at least one wireless communication network. The time tracking devices may be configured as nodes of the wireless communication network.

The time tracking device and laboratory system provide a large number of advantages over known devices of similar kind. The time tracking device and laboratory system may allow real-time monitoring and thereby enhancing productivity, efficiency and planning reliability. For example, monitoring system performance may be possible as a paperless process. No handwritten documentation may be required that needs tedious transfer into a computer. It may be possible to obtain machine readable data in real time, less error prone and digital and more user friendly at the same time, and in particular almost time neutral for the user. Automation of otherwise manual reports, e.g. system downtime, performance, and other Key Performance Indicators may be possible. Fast and live user feedback, e.g. which machine is currently available and/or where is information from ‘booking tools’ outdated, may be available. Consistent measurement of instrument downtime may be possible, e.g.

for revealing where is it unnecessary and unplanned? Revealing unknown factors in instrument downtime may be possible, too. It may be possible to uncover hidden performance such that it may be possible instead of increasing instrument numbers rather improving performance of existing instruments. It may be possible to enhance communication and collaboration. It may be possible to switch to predictive maintenance, wherein any machine with unexpected downtime can point to root causes that have previously not been revealed. Improved planning based on real usage data may be possible. It may be possible to distinguish between instrument usage and actual instrument runtime, e.g. which are the steps that take too much time, how can usage of the instrument be further improved also from usability and hardware view? It may be possible to benchmark different systems and locations against each other by standardized measures and leading to best practices. The devices may be suitable for S2 laboratories and independent of instrument type and models. It may be possible to integrate the time tracking device into existing wireless networks, such as WiFi and/or IoT, e.g. Lora, BLE networks.

It may be possible to make updates over the air (UOA) of the time tracking device software by using the wireless networks of the tracking device such as WiFi and/or IoT, e.g. Lora, BLE. UOA may be received or pushed from another computer system to the time tracking device.

In a further aspect, a method for monitoring system performance of at least one laboratory instrument is proposed. The method comprises using at least one time tracking device according to the present invention. Thus, with respect to embodiments and definitions of the time tracking device reference is made to the description of the time tracking device above or below. The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlapping fashion. The method may comprise further method steps which are not listed. The method comprises the following steps:
i) displaying at least one status indication of the laboratory instrument by the interactive faces of the time tracking device;
ii) selecting the interactive face matching a current status of the laboratory instrument by a user via changing motion and/or orientation of the time tracking device,
iii) detecting the change of motion and/or orientation of the time tracking device by using the motion sensor;
iv) measuring time by using the electronics unit, wherein a time measurement is initiated by detecting a change of motion and/or orientation of the time tracking device and is terminated by detecting a subsequent change of motion and/or orientation of the time tracking device;
v) providing data relating to the measured time via at least one communication interface of the time tracking device.

Summarizing and without excluding further possible embodiments, the following embodiments may be envisaged:

Embodiment 1 A time tracking device for monitoring system performance of at least one laboratory instrument, wherein the time tracking device comprises at least one support structure, wherein the time tracking device comprises at least one electronics unit and at least one motion sensor housed by the support structure, wherein the motion sensor is configured for detecting a change of motion and/or orientation of the time tracking device, wherein the electronics unit is configured for measuring time, wherein a time measurement is initiated by detecting a change of motion and/or orientation of the time tracking device and is terminated by detecting a subsequent change of motion and/or orientation of the time tracking device, wherein the support structure is a polygonal support structure comprising a plurality of faces, wherein the support structure comprises at least two interactive faces, wherein each of the interactive faces comprises at least one user interface comprising at least one display device, wherein each of the interactive faces is configured for displaying at least one status indication of the laboratory instrument, wherein the interactive face matching a current status of the laboratory instrument is selectable by a user via changing motion and/or orientation of the time tracking device, wherein the time tracking device is configured for providing data relating to the measured time via at least one communication interface.

Embodiment 2 The time tracking device according to any one of the preceding embodiments, wherein the user interface is a bi-directional interface.

Embodiment 3 The time tracking device according to any one of the preceding embodiments, wherein the electronics unit is configured for assigning the measured time to a status of the laboratory instrument depending on the selected interactive face.

Embodiment 4 The time tracking device according to any one of the preceding embodiments, wherein each of the interactive faces is configured for displaying one status indication of the laboratory instrument irrespectively of a number of turns of the time tracking device.

Embodiment 5 The time tracking device according to any one of the preceding embodiments, wherein the time tracking device is configured for hyperscrolling, wherein the status indication displayed by the interactive faces depends on a number of turns of the time tracking device.

Embodiment 6 The time tracking device according to any one of the preceding embodiments, wherein the electronics unit is configured for controlling the display devices, wherein the controlling comprises adapting an orientation of information displayed by the display devices depending on orientation of the time tracking device.

Embodiment 7 The time tracking device according to any one of the preceding embodiments, wherein the status indication of the laboratory instrument displayed by the respective interactive face is assignable to the respective interactive face.

Embodiment 8 The time tracking device according to any one of the preceding embodiments, wherein the interactive face is configured for displaying one or more of at least one warning message, at least one maintenance information, information about an owner of the laboratory instrument, information about at least one further laboratory instrument, a menu, at least one readout of at least one sensor, messages from third party applications and/or sensors, data sources in general, previous status information, details from an instrument related documentation system such as Digital Instrument Book, booking status of the instrument feed by an external database such as booking system and/or activity planner.

Embodiment 9 The time tracking device according to any one of the preceding embodiments, wherein each of the faces of the support structure comprises at least one light emitter configured for emitting light in a plurality of wavelengths, wherein a wavelengths is assigned to each of the status indications, wherein the electronics unit is configured for switching the wavelengths of the light emitters depending on the interactive face selected by the user.

Embodiment 10 The time tracking device according to any one of the preceding embodiments, wherein the motion sensor comprises at least one gyrosensor.

Embodiment 11 The time tracking device according to any one of the preceding embodiments, wherein the support structure is a three dimensional structure selected from the group consisting of: a pyramid; a lying trigonal prism, a lying pentagonal prism, a lying hexagonal prism, a cube.

Embodiment 12 The time tracking device according to any one of the preceding embodiments, wherein the support structure comprises at least one polygonal solid shell, for example a plastic shell or a metal shell.

Embodiment 13 The time tracking device according to any one of the preceding embodiments, wherein the electronics unit comprises at least one memory configured for storing a time log.

Embodiment 14 The time tracking device according to any one of the preceding embodiments, wherein the time tracking device comprises at least one sensor configured for monitoring environmental conditions, wherein the sensor is selected from the group consisting of: at least one temperature sensor, at least one pressure sensor; at least one humidity sensor; at least one sensor configured for detecting magnetic interferences; at least one location sensor such as at least one GPS sensor, Glonass sensor, Galileo sensor, BLE sensor.

Embodiment 15 The time tracking device according to any one of the preceding embodiments, wherein the time tracking device comprises at least one reader, wherein the reader is configured for receiving information about a user from at least one identifier of the user, wherein the electronics unit is configured for combining the measured time with the information about the user.

Embodiment 16 The time tracking device according to any one of the preceding embodiments, wherein the time tracking device comprises at least one rechargeable energy unit, wherein the rechargeable energy unit is configured for wireless charging.

Embodiment 17 The time tracking device according to any one of the preceding embodiments, wherein the time tracking device comprises at least one speaker unit configured for providing audible information.

Embodiment 18 The time tracking device according to any one of the preceding embodiments, wherein the time tracking device comprises at least one microphone unit configured for receiving voice commands.

Embodiment 19 The time tracking device according to any one of the preceding embodiments, wherein the time tracking device is remote controllable by receiving commands via the communication interface.

Embodiment 20 The time tracking device according to any one of the preceding embodiments, wherein the time tracking device comprises at least one switch, wherein the electronic unit is configured for performing at least one predefined action triggered by activating the switch, wherein the predefined action comprises one or more of providing information that the laboratory instrument breaks down to a further device via the communication interface, switching off the time tracking device, put the time tracking device into standby, or providing an automated message to a next user and/or further device via the communication interface that the laboratory instrument is available.

Embodiment 21 A laboratory system comprising at least one laboratory instrument and at least one time tracking device according to any one of the preceding embodiments, wherein the laboratory system further comprises at least one processing device, wherein the processing device comprises at least one communication interface configured for receiving data relating to the measured time from the communication interface of the time tracking device, wherein the processing device comprises at least one evaluation device configured for monitoring system performance of the laboratory instrument by evaluating the data relating to the measured time received from the communication interface of the time tracking device.

Embodiment 22 The laboratory system according to the preceding embodiment, wherein the laboratory system comprises a plurality of laboratory instruments, wherein the laboratory system comprises a plurality of time tracking devices each assigned to one of the laboratory instruments.

Embodiment 23 The laboratory system according to the preceding embodiment, wherein the laboratory system comprises at least one wireless communication network, wherein the time tracking devices are configured as nodes of the wireless communication network.

Embodiment 24 A method for monitoring system performance of at least one laboratory instrument, wherein the method comprises using at least one time tracking device according to any one of the preceding embodiments relating to a time tracking device, wherein the method comprises the following steps:
i) displaying at least one status indication of the laboratory instrument by the interactive faces of the time tracking device;
ii) selecting the interactive face matching a current status of the laboratory instrument by a user via changing motion and/or orientation of the time tracking device;
iii) detecting the change of motion and/or orientation of the time tracking device by using the motion sensor;
iv) measuring time by using the electronics unit, wherein a time measurement is initiated by detecting a change of motion and/or orientation of the time tracking device and is terminated by detecting a subsequent change of motion and/or orientation of the time tracking device;
v) providing data relating to the measured time via at least one communication interface of the time tracking device.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1A to 1D illustrate typical dimensions (FIG. 1A) and shapes (FIGS. 1B, 1C and 1D) of embodiments of a time tracking device according to the present invention;

FIG. 2A to 2G show different views of an embodiment of the time tracking device with a hexagonal shape;

FIGS. 3A to 3E show different views of an embodiment of the time tracking device in form of a cube;

FIGS. 4A and 4B show different embodiments of the time tracking device equipped with a hyperscrolling feature;

FIGS. 5A to 5D show a flow chart illustrating the use of a time tracking device (FIG. 5A), the time tracking device in corresponding situations (FIGS. 5B and 5C) and a user interacting with the time tracking device; and

FIG. 6 shows part of an embodiment of a laboratory system according to the present invention.

### DETAILED DESCRIPTION OF THE EMBODIMENTS

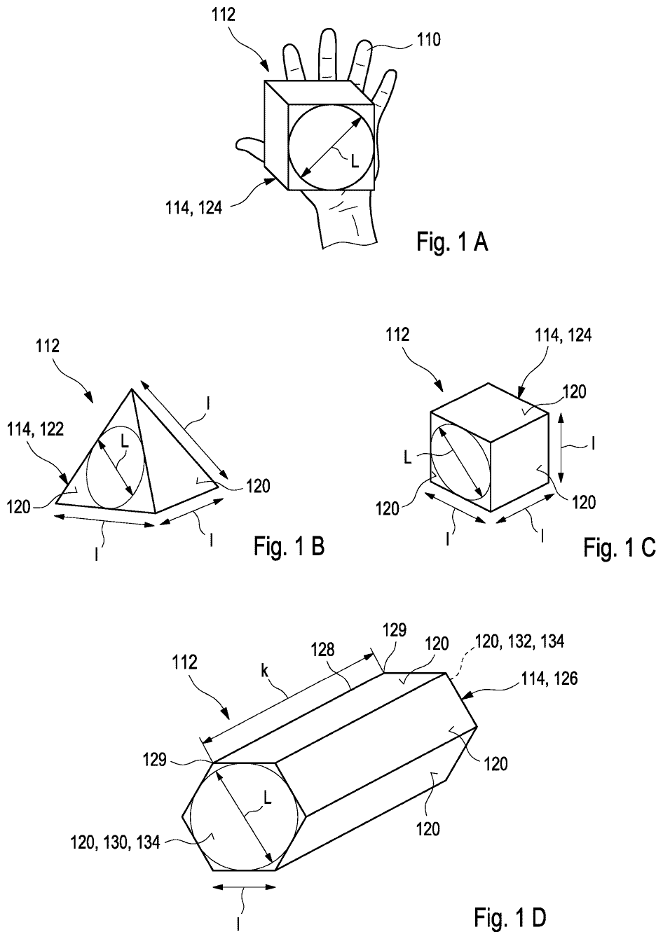

FIG. 1A shows an embodiment of a time tracking device 112 for monitoring system performance of at least one laboratory instrument according to the present invention. The laboratory instrument may be an arbitrary device of a laboratory configured for performing at least one function such as analysis of the biological sample. The laboratory instrument may be one or more of a pre-analytic laboratory instrument, an analytic laboratory instrument, or a post-analytic laboratory instrument. The laboratory may be a location configured for work in the field of the natural sciences and/or engineering in the sense that it offers the opportunity to conduct corresponding measurements and controls. The laboratory may be designed as in-vitro diagnostics laboratory configured for performing at least one test on a sample, e.g. a biological sample that has been taken off the human body or animal body. For example, the test on the sample may comprise applying at least one reagent to the sample and monitoring a detectable reaction. For example, the laboratory may be a clinical laboratory, a medical laboratory, a forensic laboratory or a blood bank.

The time tracking device 112 may be configured for performing time tracking. The time tracking device 112 may be configured for measuring a time duration of a status of the laboratory instrument. The time tracking device 112 may comprise at least one timer or clock for measuring time. The timer or clock may be electronically driven. The time tracking device 112, additionally or alternatively, may be designed for receiving an external time signal such as from an atomic clock via a satellite television or computer connection. The time tracking device 112 may be configured for measuring time with a precision in ms or us range.

The time tracking device 112 is configured for monitoring system performance. The system performance may be information about times of use, information about availability, information about system downtime such as information about repair times, information about maintenance times, information about times the laboratory instrument is out of order, information about times the laboratory instrument is offline. The monitoring of the system performance may comprise determining system performance of the laboratory instrument at at least two different time points such as over a period of time. The monitoring may comprise one or more of documenting the system performance, determining at least one time log, preparing at least one report about the system performance such as a manual report.

The time tracking device comprises at least one support structure 114. The support structure 114 may be an arbitrary shaped three-dimensional body. The support structure 114 may be an element which is configured for fully or partially enclosing and/or covering one or more components and for providing protection for these one or more components, such as against environmental influences and/or mechanical influence and/or humidity. The support structure 114 may be configured as mounting base, which may also be referred to as mounting frame 150, and/or housing.

The support structure 114, specifically, may be a rigid support structure made of one or more of a plastic material, a metallic material or a cardboard material. For example, the support structure 114 may comprise at least one polygonal solid shell, for example a plastic shell or a metal shell. For example, the support structure 114 may comprise cutouts and/or windows 156 in which a user interface 138 may be insertable such that screens 158 of a display device 140 are visible from outside of the time tracking device 112.

The support structure 114 may be a three dimensional structure selected from the group consisting of: a pyramid 122; a lying trigonal prism, a lying pentagonal prism, a lying hexagonal prism 126, a cube 124. The time tracking device 112 may be a mobile and/or portable time tracking device 112. As shown in FIG. 1A, the dimensions of the time tracking device 112 may be adapted to a diameter of a hand 110 of an average adult. In the embodiment shown in FIG. 1A, the time tracking device 112 has the shape of a cube 124. The diameter of the hand 110 and the dimension of the time tracking device 112 of corresponding size is indicated with a double-sided arrow marked “L” in FIGS. 1A to 1D,. For example, the dimension marked “L” of the time tracking device 112 may be in a range from 5 cm to 10 cm. A length of an edge 128 of the time tracking device 112 marked “I” may be adapted accordingly, as can be seen in FIGS. 1B to 1D.

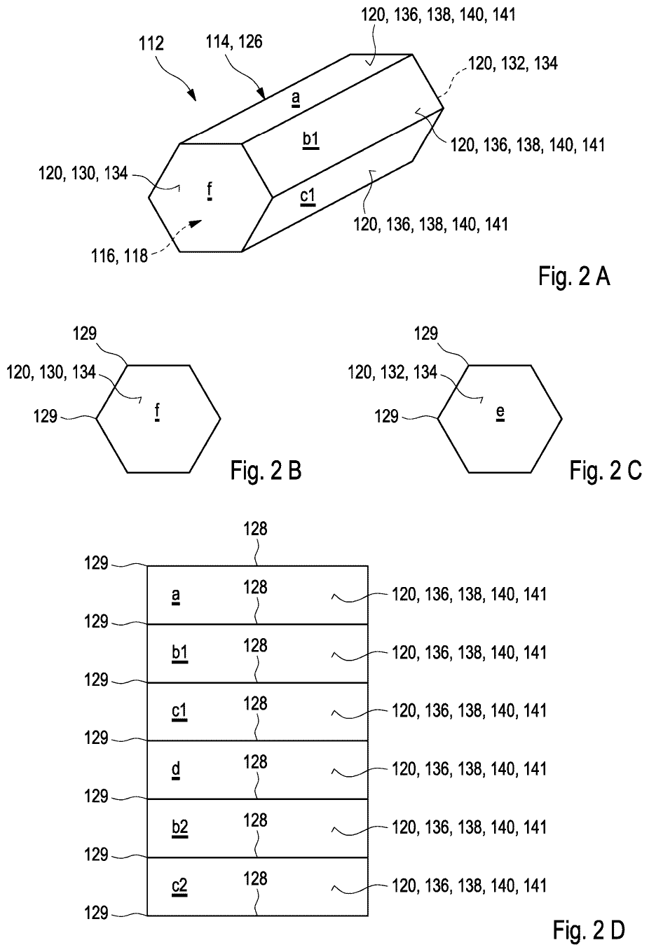

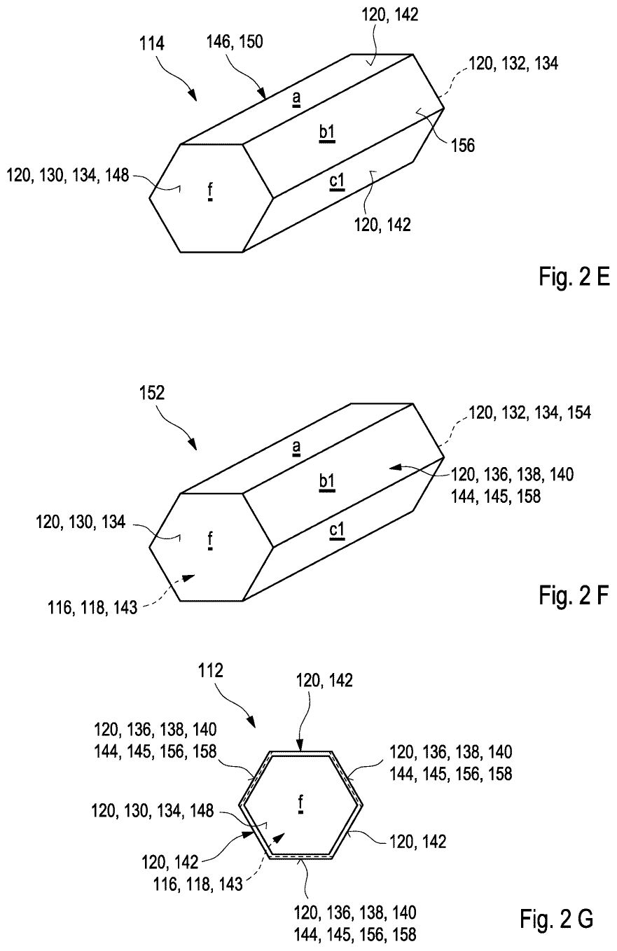

The time tracking device 112 comprises at least one electronics unit 116 and at least one motion sensor 118 housed by the support structure 114, see e.g. FIG. 2.

The electronics unit 116 may be at least one unit of the time tracking device 112 configured for one or more of performing the time measurement, storing data, processing data, providing power to further components of the time tracking device 112 and the like. The electronics unit 116 may comprise at least one processor, such as at least one microprocessor. The processor may be an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric coprocessor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor such as a Raspberry Pi® or Arduino® based microprocessor. Specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) or the like. The processor specifically may be configured, such as by software programming, for performing one or more evaluation operations.

The motion sensor 118 may be a sensor device having at least one sensor element configured for detecting a change of motion and/or orientation of the time tracking device 112. The change of motion may be an acceleration along at least one axis such as in a horizontal plane, in a vertical plane and/or around an axis of rotation. For example, the change of motion may comprise a shaking of the time tracking device 112. The change of orientation may comprise at least one movement of the time tracking device 112 around an axis of rotation. The change of orientation may comprise for example rotating and/or flipping and/or scrolling of the time tracking device 112. For example, the motion sensor 118 may comprise at least one gyrosensor.

The electronics unit 116 is configured for measuring time. The electronics unit 116 may comprise at least one timer or clock for measuring time. The timer or clock may be electronically driven. The electronics unit 116, additionally or alternatively, may be designed for receiving an external time signal such as from an atomic clock via a satellite television or computer connection. The electronics unit 116 may control the time measurement. A start of the time measurement may be initiated by the motion sensor detecting a change of motion and/or orientation of the time tracking device 112. The motion sensor 118 may be configured for providing a sensor signal to the electronics unit 116. The electronics unit 116 may be configured upon receiving the sensor signal to start the time measurement. The time measurement may be terminated by the motion sensor 118 detecting a subsequent change of motion and/or orientation of the time tracking device 112 and providing a further sensor signal to the electronics unit 116. The electronics unit 116 may be configured upon receiving the sensor signal to end the time measurement.

The electronics unit 116 may comprise at least one memory configured for storing a time log. The memory may be a storage medium may refer to a non-transitory data storage means, such as a hardware storage medium having stored thereon computer-executable instructions. The memory may be or may comprise a storage medium such as a randomaccess memory (RAM) and/or a read-only memory (ROM). The time log may be a protocol comprising time-resolved recording of events, in particular relating to status changes of the laboratory instrument.

The support structure 114 is a polygonal support structure 114 comprising a plurality of faces 120. The polygonal support structure 114 may be embodied in numerous different three-dimensional structures, such as those shown in an exemplary fashion in FIGS. 1B, 1C and 1D. Thus, the polygonal support structure 114 may be a pyramid 122, a cube 124 or a hexagonal prism 126, as shown in Figures IB, IC and ID respectively.

Polygonal support structures 114 of other shapes are feasible. In case of the polygonal support structure 114 being embodied as a hexagonal prism 126 as shown in FIG. 1C, a lateral edge 128 connecting two corners 129 of a hexagon 134 may have a dimension in the range of 8 cm to 20 cm. This dimension is marked “k” in FIG. 1D.

FIG. 2A shows an embodiment of the time tracking device 112 with the support structure 114 in the form of a lying hexagonal prism 126. The support structure 114 of the time tracking device 112 shown in FIG. 2A is a lying hexagonal prism 126 with eight faces 120, which are further illustrated in FIGS. 2B, 2C and 2D. FIGS. 2B and 2C show one face 120 each corresponding to a front 130, marked “f”, and a back 132, marked “e”, respectively. Both the front 130 and the back 132 have the shape of a hexagon 134. FIG. 2D illustrates the remaining six faces 120, which together form a circumferential surface of the lying hexagonal prism 126 of FIG. 2A, when face 120 “c2” and face 120 “a” are joint. For illustrative purposes the circumferential surface is shown in FIG. 2D in a cut open and unfolded manner with the six faces 120 side by side. In FIGS. 2A, 2B, 2C and 2D each face 120 shown in the respective Figure is marked. The front face 120 is marked “f”, the back face 120 is marked “e”, the faces 120 forming the circumferential surface are marked “a” for top, “d” for bottom, “b1” and “b2” for faces 120 pointing upwards and “c1” and “c2” for the faces 120 pointing downwards, corresponding to a position of each face 120 in FIG. 2A, which shows a perspective view of the entire time tracking device 112.

The support structure 114 comprises at least two interactive faces 136, wherein each of the interactive faces 136 comprises at least one user interface 138 comprising at least one display device 140. An interactive face 136 may be a face 120 of the support structure 114 configured for providing a uni- or bi-directional interface for a user 137 of the time tracking device 112. The user interface 138 may be configured for interacting with its environment, such as for the purpose of unidirectionally or bidirectionally exchanging information, such as for exchange of one or more of data or commands. For example, the user interface 138 may be a bi-directional interface. The user interface 138 may be configured to share information with a user 137 and to receive information by the user 137. The user interface 138 may be configured for visually interacting with a user 137. Therefore, the user interface 138 comprises the at least one display device 140.

The display device 140 may be at least one electronic device comprising at least one display. The display may be configured for displaying an item of information such as at least one image, at least one diagram, at least one histogram, at least one text, at least one sign. Specifically, the display device 140 comprises the at least one screen 158. The screen 158 may have an arbitrary shape such as a rectangular shape. The shape of the screen 158 may be adapted to the shape of the support structure 114. The display device 140 may comprise one or more of a liquid crystal display (LCD), a thin-film transistor (TFT) display, a light emitting diode (LED) display, an organic light emitting diode (OLED) display, in particular an OLED foil display, an eINK® display. The display device 140 may be touch sensitive and/or gesture sensitive. For example, the display device 140 may be configured such that simple gestures trigger one or more actions of the time tracking device, e.g. shaking to start time measurement, tapping twice to go back to a home screen, and the like. The time tracking device 112 may comprise electrical connections between the display device 140 and the electronics unit 116, e.g. for controlling the display device 140 via the electronics unit 116, providing power to the display device 140 and the like.

Each of the interactive faces 136, via the display device 140, may be configured for displaying at least one status indication 171 of the laboratory instrument. The interactive face 136 matching a current status of the laboratory instrument is selectable by a user 137 via changing motion and/or orientation of the time tracking device 112. The status of the laboratory instrument may be one or more of in use, standby, maintenance, repair, out of order, offline. The status indication 171 may be an arbitrary visual indication. The visual indication may comprise using light of a specific wavelength indicating a status of the laboratory instrument, text and/or numbers and/or characters indicating a status of the laboratory instrument.

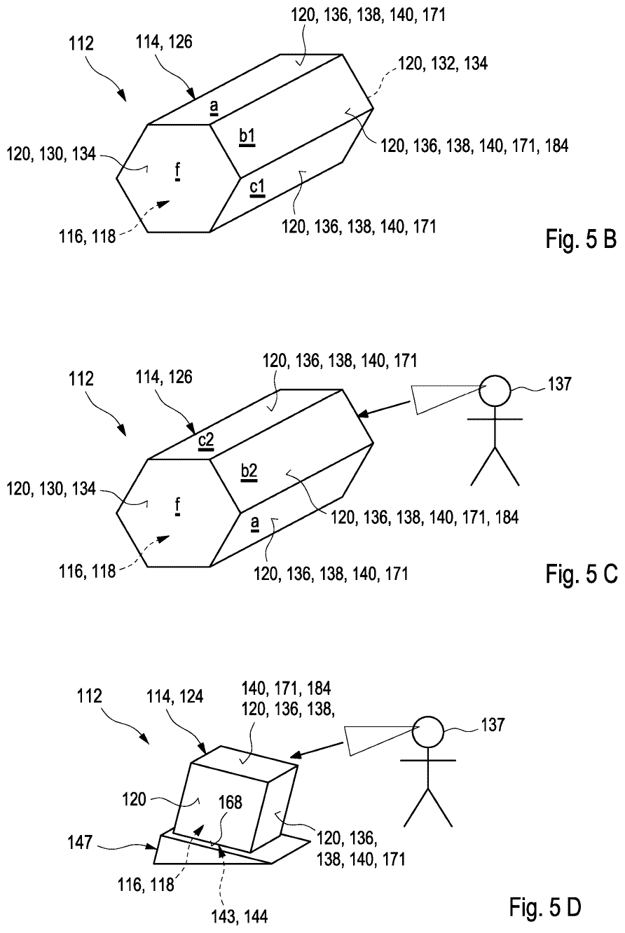

The status indication 171 of the laboratory instrument displayed by the respective interactive face 136 may be assignable to the respective interactive face 136. The electronics unit 116 may be configured for controlling, e.g. setting, the status indication 171 displayed by the interactive faces 136. The electronics unit 116 may be controllable by a remote control, such as via at least one computer connected with the time tracking device 112, such that the status indication 171 may be assignable by the remote control. For example, the status indication 171 may be assigned depending on the type of laboratory instrument and/or history of laboratory instrument. The current status may be an actual status of the laboratory instrument. The interactive face 136 matching a current status of the laboratory instrument is selectable by a user 137 via changing motion and/or orientation of the time tracking device 112. For example, the time tracking device 112 may comprise at least one selection position 184, e.g. shown in the embodiment of FIG. 5. The time tracking device 112 may be configured for selecting a current status of the laboratory instrument by bringing the interactive face 136 corresponding the current status of the laboratory instrument into the selection position 184. The selection position 184 may be defined with respect to direction of the gravitational force. The selection position 184 may be a position in which the interactive face 136 to be selected is oriented upwards, i.e. the direction of the gravitational force being a normal of a plane of the interactive face 136. However, other selection positions may be possible. The selection position 184 may be defined depending on the three dimensional structure of the support structure 114. For example, in case of a cube 124 the selection position 184 may be a position in which the interactive face 136 to be selected is oriented upwards. For example, in case of a lying hexagonal prism 126, the selection position 184 may be a position in which the interactive face 136 to be selected forms an angle of 30° with the direction of the gravitational force. The selection position 184 may be defined depending on a surface 168 the time tracking device 112 is arranged on such as a surface 168 of a charging element 147. The charging element 147 may also be referred to as a charging station 147. The orientation with respect to the direction of the gravitational force may be determined by the motion sensor 118 such as the gyrosensor. Additionally or alternatively, the status indication 171 matching to a current status of the laboratory instrument may be selected by using at least one gesture such as tapping on an interactive face 136. The electronics unit 116 may be configured for assigning the measured time to a status of the laboratory instrument depending on the selected interactive face 136.

The electronics unit 116 may be configured for controlling the display devices 140. As outlined above, the time tracking device 112 may be moved such as turned or flipped. Specifically, the controlling may comprise adapting an orientation of information displayed by the display devices 140 depending on orientation of the time tracking device 112. This may allow user friendly reading of the status indication 171 by a user 137.

The interactive faces 136 may be configured for showing additional information. The interactive faces 136 may be configured for displaying one or more of at least one warning message, at least one maintenance information, information about an owner of the laboratory instrument, information about at least one further laboratory instrument, a menu, at least one readout of at least one sensor, messages from third party applications and/or sensors, data sources in general, previous status information, details from an instrument related documentation system such as Digital Instrument Book, booking status of the instrument feed by an external database such as booking system and/or activity planner. For example, the interactive faces 136 may be configured for displaying cross information from further time tracking devices 112 and/or laboratory instruments of the laboratory, e.g. for showing errors of other analyzers in near proximity. This may allow not showing only a limited number of information to a user 137 but information relating to usage of the laboratory instrument.

Each of the faces 120 of the support structure 114 may comprise at least one light emitter 141 configured for emitting light in a plurality of wavelengths. The light emitter 141 may be at least one light emitting diode (LED) 142. For example, in case of the interactive faces 136 the light emitter 141 may be the display device 140 and/or at least one further light emitter 141 such as an LED 142. A wavelength may be assigned to each of the status indications 171, wherein the electronics unit 116 may be configured for switching the wavelengths of the light emitters 141 depending on the interactive face 136 selected by the user 137.

FIGS. 2E, 2F and 2G show a particular embodiment of the time tracking device 112 in the shape of a lying hexagonal prism 126. FIG. 2E shows the support structure 114 comprising an outer framework 146 with front cover 148. FIG. 2F shows an electronic insert 152 slidable into the framework 146. FIG. 2G shows a front view of the time tracking device 112 with the electronic insert 152 slided into the outer framework 146. In this embodiment, the faces 120 of the circumferential surface each comprise either at least one light emitting diode (LED) 142 or the display device 140 in combination with a connector 144 for charging a rechargeable energy unit 143. The LEDs 142 may be arranged at the faces 120 marked “a”, “c1” and “b2”. The combination of the display device 140 and the connector 144 may be arranged at the faces 120 marked “b1”, “d” and “c2”. The faces “a”, “b1” and “c1” are visible in the perspective view shown in FIG. 2E, while the faces “d”, “b2” and “c” are hidden. The order of the faces “a”, “b”, “c1”, “d”, “c2” and “b2” is the same as shown for the embodiment illustrated in FIGS. 2A to 2D. The connector 144 may in particular comprise at least one inductive, e.g. qi charger 145, wherein the qi charger 145 is placed on a charging station 147 for charging the rechargeable energy unit 143. The support structure 114 may comprise an outer framework 146 with a front cover 148. The outer framework 146 may comprise a mounting frame 150 for mounting an electronic insert 152. The electronic insert 152 may be slidable into the outer framework 146. The electronic insert 152 may comprise a back cover 154 as indicated in FIG. 2F. The electronic insert 152 may comprise the display device 140, particularly three display devices 140 as shown in FIG. 2G, as well as at least one processor and graphics (not shown). The support structure 114, in particular the outer framework 146, may comprise windows 156, also referred to as cut-outs, such that screens 158 of the display devices 140 may be visible from the outside of the time tracking device 112 when the electronic insert 152 is slided into the outer framework 146. FIG. 2G shows a front view of this embodiment of the time tracking device 112 where faces 120 with LEDs 142 alternate with faces 120 comprising display devices 140 and qi chargers 145. The display devices 140 are indicated by dashed lines in FIG. 2G.

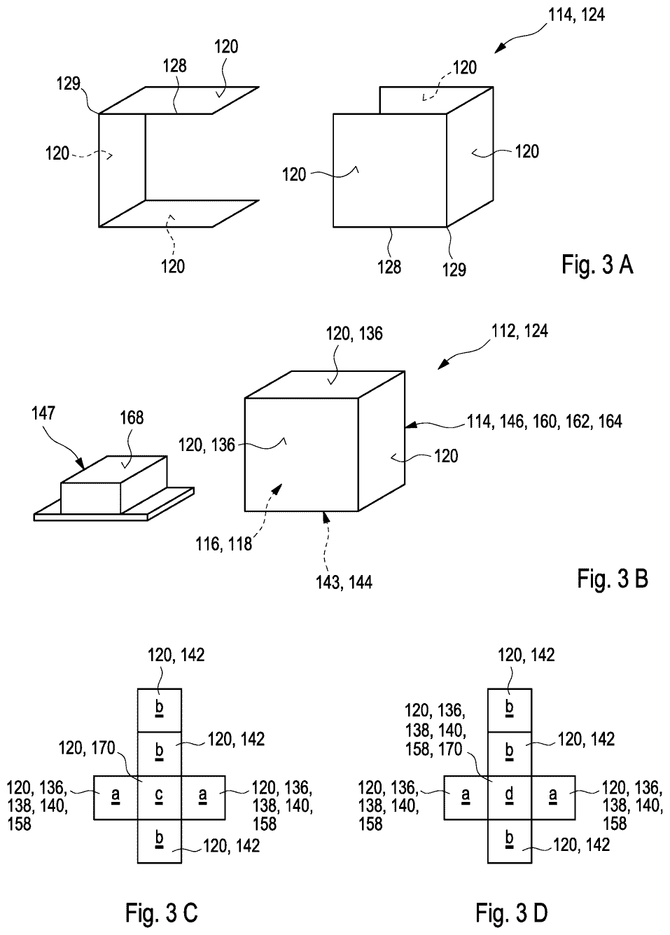

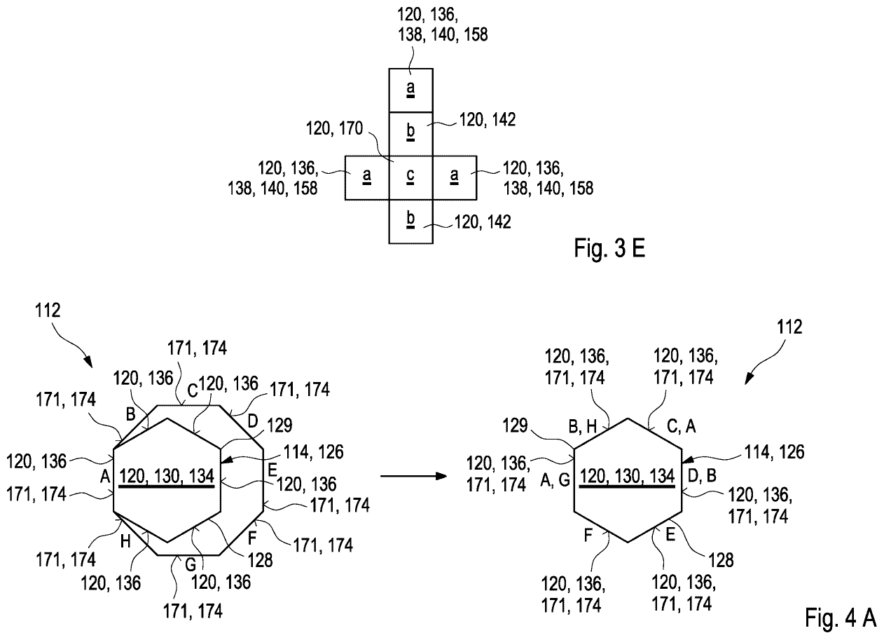

FIGS. 3A to 3E show different aspects of a further embodiment of the time tracking device 112 in the shape of a cube 124. In the embodiment shown in FIGS. 3A to 3E, the time tracking device 112 may comprise at least one sensor 160 configured for monitoring environmental conditions. The sensor 160 may be selected from the group consisting of: at least one temperature sensor, at least one pressure sensor; at least one humidity sensor; at least one sensor configured for detecting magnetic interferences; at least one location sensor such as at least one GPS sensor, Glonass sensor, Galileo sensor, at least one Bluetooth low energy (BLE) sensor. The sensor 160 may monitor the environmental conditions such as temperature, pressure, humidity. The sensor 160 may in particular be integrated into the support structure 114, e.g. in the outer framework 146, as indicated in FIG. 3B. The time tracking device 112 may further comprise at least one speaker unit 162 configured for providing audible information as also shown in FIG. 3B.

The time tracking device 112 may further comprise at least one microphone unit 164 configured for receiving voice commands. Speaker unit 162 and microphone unit 164 may allow using the time tracking device 112 as laboratory journal such as for dictating and documentation. This may allow “Hands free documentation”.

The time tracking device 112 may further comprise the connector 144 for charging the rechargeable energy unit 143. For charging, the time tracking device 112 may be arranged on a surface 168 of the charging station 147.

FIGS. 3C, 3D and 3E each show the six faces 120 of the cube-shaped embodiment of the time tracking device 112 in a cut open and unfolded manner with each of the six faces 120 being marked with either “a”, “b”, “c” or “d”. FIGS. 3C, 3D and 3E thus illustrate possible arrangements of the screens 158 of the display devices 140 corresponding to marking “a”, of the LEDs 142 or OLEDs corresponding to marking “b”, of a baseplate 170 without screen 158 corresponding to marking “c” and a baseplate 170 with screen 158 corresponding to marking “d”. Other arrangements are feasible.

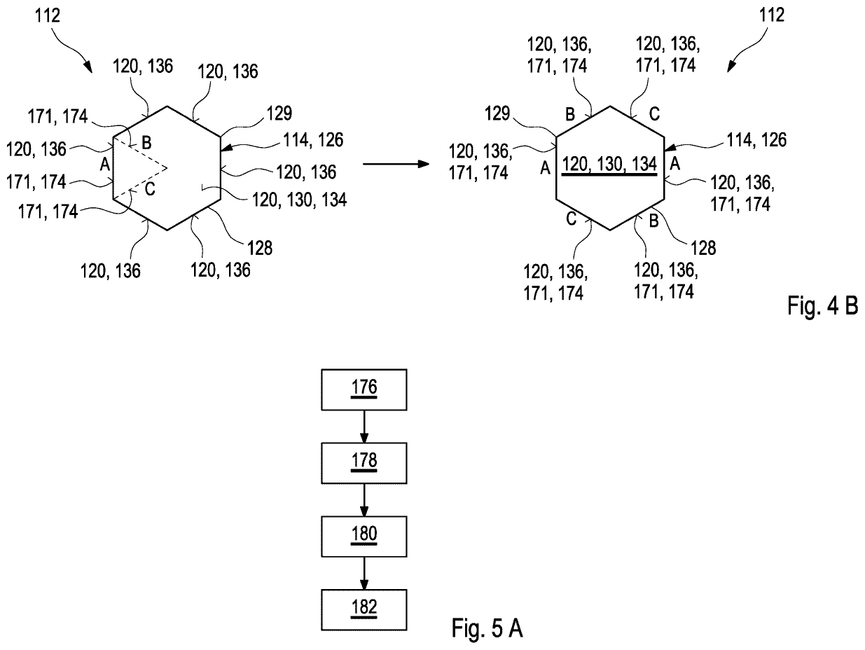

FIGS. 4A and 4B show a front view of an embodiment of the time tracking device 112 with the support structure 114 having the shape of a hexagonal prism 126. Thus, the support structure 114 as shown in FIGS. 4A and 4B comprises eight faces 120.

FIG. 4A shows an embodiment, wherein the time tracking device 112 may be configured for hyperscrolling. The status indication 171 displayed by the interactive faces 136 may depend on a number of turns of the time tracking device 112. A number of virtual screens 174, which may also be referred to as pages, may exceed the number of faces 120, in particular interactive faces 136, of the support structure 114. Thus, the support structure 114 may be a lying hexagonal prism 126 offering six interactive faces 136, as indicated in FIG. 4A, as well as a front 130 and a back 132, which are not embodied as interactive faces 136. At the same time the time tracking device 112 may allow selecting 6+x different status indications 171, corresponding to 6+x virtual screens 174. In the embodiment shown in FIG. 4A eight such different status indications 171 are foreseen and marked “A”, “B”, “C”, “D”, “E”, “F”, “G” and “H”. FIG. 4A further shows a possible assignment of the eight different status indications 171 to the six interactive faces 120 of the time tracking device 112. The status indications 171 may become visible through overtwisting the time tracking device 112.

In the embodiment of FIG. 4B, each of the interactive faces 136 may be configured for displaying one status indication of the laboratory instrument irrespectively of a number of turns of the time tracking device 112. The time tracking device 112 may have fewer virtual screens 174 than faces 120, in particular interactive faces 136, of the support structure 114. As shown in FIG. 4B, the time tracking device 112 may comprise six interactive faces 120 and three virtual screens 174 corresponding to three status indications 171 marked “A”, “B” and “C”. FIG. 4B further shows a possible assignment of the three different status indications 171 to the six interactive faces 120 of the time tracking device 112. Further, a usage of the hyperscrolling feature may be effectable via simple actions by the user 137 such as double tapping for scrolling forward, triple tapping for scrolling backward and shaking to go back to home screen.

FIG. 5A shows a flow chart illustrating the use of the time tracking device 112, while FIG. 5B shows the time tracking device 112 and a user 137 in corresponding situations. The flow chart shows the following steps and situations: “The face marked “a” of time tracking device 112 is facing upward” (reference number 176); “The time tracking device 112 is rotated clockwise by 120°” (reference number 178); “b2” and “c1” are rotated by 180° “(reference number 180); and” LED changes colors accordingly and simultaneously“(reference number 182). These steps and situations are described in more detail below.

As indicated in step 176 of FIG. 5A, an exemplary sequence of situations and actions regarding the time tracking device 112 may start with the time tracking device 112 in the shape of a lying hexagonal prism 126 being situated with the face 120 marked “a” facing upward. This situation is illustrated in FIG. 5B. The time tracking device 112 may comprise the at least one selection position 184. The time tracking device 112 may be configured for selecting a current status of the laboratory instrument by bringing the interactive face 136 corresponding to the current status of the laboratory instrument into the selection position 184. For example, in case of the lying hexagon 126, the selection position 184 may be a position in which the interactive face 136 to be selected forms an angle of 30° with the direction of the gravitational force. In FIG. 5B, the face 120 marked “b1” may be in the selection position 184. It may be an interactive face 136 comprising the user interface 138. The user interface 138 of face “b1” may display the status indication 171 of the laboratory instrument, e.g. “stand-by”, via the display device 140.

The time tracking device 112 may then be rotated by the user 137, e.g. by 120° clockwise as described in step 178. The clockwise rotation becomes apparent by comparing the perspective views of the time tracking device 112 shown in FIGS. 5B and 5C. The motion sensor 118 may be configured for detecting a change of motion and/or orientation of the time tracking device 112, e.g. the clockwise rotation by 120° effected by the user 137. The electronics unit 116 is configured for measuring time, wherein a time measurement is initiated by detecting a change of motion and/or orientation of the time tracking device 112. Thus, the time measurement may be started by the clockwise rotation by 120° effected by the user 137, who may select by the rotational movement the further interactive face 136 displaying the status indication 171 “in use” of the laboratory instrument. Said further interactive face 136 arranged in the selection position 184 after the clockwise rotation by 120° is marked “b2” in FIG. 5C. The selection position 184 may be chosen such that it may be easily grasped by the user 137 as shown in FIG. 5C. The motion sensor 118 may comprise at least one gyrosensor for determining the orientation of the time tracking device 112 with respect to the direction of the gravitational force. The electronics unit 116 may be configured for adapting an orientation of information displayed by the display devices 140 depending on an orientation of the time tracking device 120, e.g. by rotating the displayed information for “b2” and “c1” by 180° and for “f” by 120° such that e.g. a Logo is oriented upwards. This is indicated in step 180. Upon changing the orientation of the time tracking device 112 by rotation, the LEDs 142 may change colors accordingly and ideally simultaneously as indicated in step 182. The user 137 may use the laboratory instrument e.g. for a measurement that may take a certain period of time. The time measurement is terminated by detecting a subsequent change of motion and/or orientation of the time tracking device 112. Thus, having finished the measurement, the user 137 may terminate the time measurement by a further rotation of the time tracking device 112. Thus, the user 137 may rotate the time tracking device 112 to select the status indication 171 “stand-by” again. Other options matching further status of the laboratory instrument may also be selectable, such as maintenance, maintenance, repair, out of order, offline. The time tracking device 112 is configured for providing data relating to the measured time via at least one communication interface 172.

FIG. 5D shows a user 137 interacting with the time tracking device 112 in the shape of a cube 124. The time tracking device 112 may comprise the rechargeable energy unit 143 e.g. configured for wireless charging. The rechargeable energy unit 143 may comprise at least one rechargeable battery. The rechargeable energy unit 143 may be part of the electronics unit 116 or may be a separate unit. For example, the rechargeable energy unit 143 may comprise at least one battery housing configured for receiving at least one rechargeable battery. The rechargeable energy unit 143 may be configured for wireless charging. The time tracking device 112 may comprise the at least one connector 144 for charging the rechargeable energy unit 143. The connector 144 may be arranged at a face 120 of the support structure 114. For example, the time tracking device 112 may comprise at least one qi charger 145, wherein the qi charger 145 is placed on a charging station 147 for charging the rechargeable energy unit 143. The time tracking device 112 may comprise a plurality of connectors 144, in particular a plurality of qi chargers 145. Specifically, each of the faces 120 of the time tracking device 112 may comprise one of the qi chargers 145. The qi charger 145 may also be arranged underneath the screen 158, e.g. in the case that the interactive faces 136 comprise the qi chargers 145. In FIG. 5D the time tracking device 112 is arranged on the surface 168 of the charging station 147 for wireless charging. The surface 168 of the charging station 147 on which the time tracking device 112 is arranged for charging may be an inclined surface 168. The inclined surface 168 may define the selection position 184. The slope of the inclined surface 168 may define a reading direction of a user 137 as illustrated in FIG. 5D.

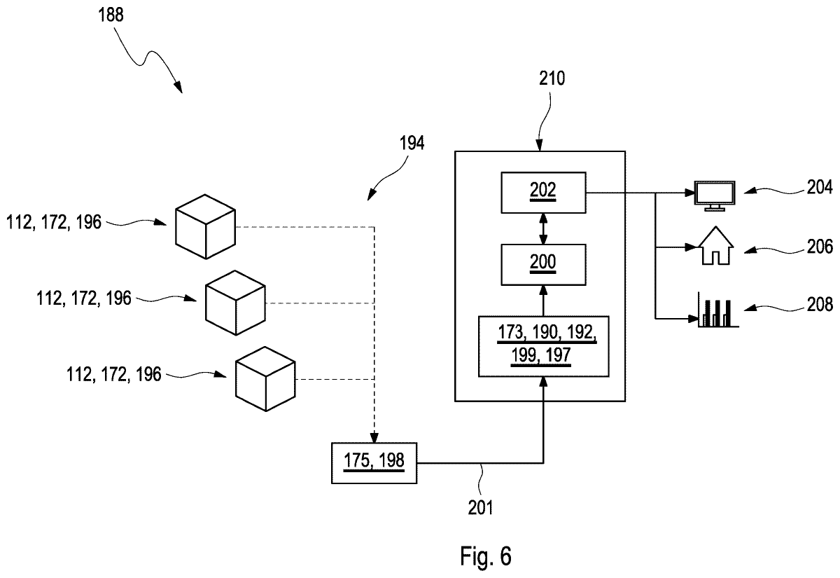

FIG. 6 shows part of an embodiment of a laboratory system 188 according to the present invention. In this embodiment, three cube-shaped time tracking devices 112 are used. The embodiment of the laboratory system 188 illustrated in FIG. 6 further comprises three laboratory instruments (not shown), wherein each of the three time tracking devices 112 is assigned to one of the laboratory instruments. The laboratory system 188 further comprises at least one processing device 190 having at least one communication interface 173 configured for receiving data relating to the measured time from the communication interface 172 of the time tracking device 112. The processing device 190 comprises at least one evaluation device 192 configured for monitoring system performance of the laboratory instrument by evaluating the data relating to the measured time received from the communication interface 172 of the time tracking device 112.

The laboratory system 188 may furthermore comprise at least one wireless communication network 194, wherein the time tracking devices 112 are configured as nodes 196 of the wireless communication network 194. Specifically, the three time tracking devices 112 may send data relating to the measured time via their communication interfaces 172 to a beacon 198 configured for receiving the data via a communication interface 175. The data may be sent via Wi-Fi or any other wireless or wired data transfer option as indicated by dotted lines in FIG. 6. The data collected by the beacon 198 may be transferred to a computer 199, e.g. via a USB connection 201. The computer 199 may comprise a storing device 197, e.g. a hard drive for storing the data. The computer may also comprise the processing device 190 and the evaluation device 192 of the laboratory system 188. The computer 199 may also be referred to as a bridge PC. The data originally provided by the time tracking devices 112 may then be shared or made available to further devices 210 e.g. for data processing and analysis. Data processing may in particular take place using a tableau server 202 or any other database server or storage device. Processed data may be made available at a variety of sites and/or for a plurality of users, as illustrated in FIG. 6, such as in laboratories of the laboratory system, e.g. via laboratory screens 204, at internet web sites 206, or at workstations 208, where further processing and data analysis may take place.

### LIST OF REFERENCE NUMBERS

110 hand
112 time tracking device
114 support structure
116 electronics unit
118 motion sensor
120 face
122 pyramid
124 cube
126 hexagonal prism
128 edge
129 corner
130 front
132 back
134 hexagon
136 interactive face
137 user
138 user interface
140 display device
141 light emitter
142 LED
143 rechargeable energy unit
144 connector
145 qi charger
146 outer framework
147 charging station
148 front cover
150 mounting frame
152 electronic insert
154 back cover
156 window
158 screen
160 sensor
162 speaker unit
164 microphone unit
168 surface
170 baseplate
171 status indication
172 communication interface
173 communication interface
174 virtual screen
175 communication interface
176 The face marked “a” of time tracking device 112 is facing upward.
178 The time tracking device 112 is rotated clockwise by 120°.
180 “b2” and “c1” are rotated by 180°.
182 LED changes colors accordingly and simultaneously.
184 selection position
188 laboratory system
190 processing device
192 evaluation device
194 wireless communication network
196 node
197 storing device
198 beacon
199 computer
200 shared virtual space
201 USB connection
202 tableau server
204 laboratory screen
206 web site
208 workstation
210 further device

1. A time tracking device for monitoring system performance of at least one laboratory instrument, wherein the time tracking device comprises at least one support structure, wherein the time tracking device comprises at least one electronics unit and at least one motion sensor housed by the support structure, wherein the motion sensor is configured for detecting a change of motion and/or orientation of the time tracking device, wherein the electronics unit is configured for measuring time, wherein a time measurement is initiated by detecting a change of motion and/or orientation of the time tracking device and is terminated by detecting a subsequent change of motion and/or orientation of the time tracking device, wherein the support structure is a polygonal support structure comprising a plurality of faces, wherein the support structure comprises at least two interactive faces, wherein each of the interactive faces comprises at least one user interface comprising at least one display device, wherein each of the interactive faces is configured for displaying at least one status indication of the laboratory instrument, wherein the interactive face matching a current status of the laboratory instrument is selectable by a user via changing motion and/or orientation of the time tracking device, wherein the electronics unit is configured for assigning the measured time to a status of the laboratory instrument depending on the selected interactive face, wherein the status of the laboratory instrument is one or more of in use, standby, maintenance, repair, out of order, offline, wherein the time tracking device is configured for providing data relating to the measured time via at least one communication interface.

2. The time tracking device according to claim 1, wherein the user interface is a bi-directional interface.

3. The time tracking device according to claim 1, wherein each of the interactive faces is configured for displaying one status indication of the laboratory instrument irrespectively of a number of turns of the time tracking device.

4. The time tracking device according to claim 1, wherein the time tracking device is configured for hyperscrolling, wherein the status indication displayed by the interactive faces depends on a number of turns of the time tracking device.

5. The time tracking device according to claim 1, wherein the electronics unit is configured for controlling the display devices, wherein the controlling comprises adapting an orientation of information displayed by the display devices depending on orientation of the time tracking device.

6. The time tracking device according to claim 1, wherein the status indication of the laboratory instrument displayed by the respective interactive face is assignable to the respective interactive face.

7. The time tracking device according to claim 1, wherein the interactive face is configured for displaying one or more of at least one warning message, at least one maintenance information, information about an owner of the laboratory instrument, information about at least one further laboratory instrument, a menu, at least one readout of at least one sensor, messages from third party applications and/or sensors, data sources in general, previous status information, details from an instrument related documentation system, booking status of the instrument feed by an external database.

8. The time tracking device according to any one of the preceding claim 1, wherein each of the faces of the support structure comprises at least one light emitter configured for emitting light in a plurality of wavelengths, wherein a wavelengths is assigned to each of the status indications, wherein the electronics unit is configured for switching the wavelengths of the light emitters depending on the interactive face selected by the user.

9. The time tracking device according to claim 1, wherein the motion sensor comprises at least one gyrosensor.

10. The time tracking device according to any one of the preceding claim 1, wherein the support structure is a three dimensional structure selected from the group consisting of: a pyramid, a lying trigonal prism, a lying pentagonal prism, a lying hexagonal prism, a cube.

11. The time tracking device according to claim 1, wherein the electronics unit comprises at least one memory configured for storing a time log.

12. The time tracking device according to claim 1, wherein the time tracking device comprises at least one sensor configured for monitoring environmental conditions, wherein the sensor is selected from the group consisting of: at least one temperature sensor, at least one pressure sensor; at least one humidity sensor; at least one sensor configured for detecting magnetic interferences; at least one location sensor such as at least one GPS sensor, Glonass sensor, Galileo sensor, BLE sensor.

13. The time tracking device according to claim 1, wherein the time tracking device is remote controllable by receiving commands via the communication interface.

14. A laboratory system comprising at least one laboratory instrument and at least one time tracking device according to claim 1, wherein the laboratory system further comprises at least one processing device, wherein the processing device comprises at least one communication interface configured for receiving data relating to the measured time from the communication interface of the time tracking device, wherein the processing device comprises at least one evaluation device configured for monitoring system performance of the laboratory instrument by evaluating the data relating to the measured time received from the communication interface of the time tracking device.

15. A method for monitoring system performance of at least one laboratory instrument, wherein the method comprises using at least one time tracking device according to claim 1 relating to a time tracking device, wherein the method comprises the following steps:
i) displaying at least one status indication of the laboratory instrument by the interactive faces of the time tracking device;
ii) selecting the interactive face matching a current status of the laboratory instrument by a user via changing motion and/or orientation of the time tracking device;
iii) detecting the change of motion and/or orientation of the time tracking device by using the motion sensor;
iv) measuring time by using the electronics unit, wherein a time measurement is initiated by detecting a change of motion and/or orientation of the time tracking device and is terminated by detecting a subsequent change of motion and/or orientation of the time tracking device;
v) providing data relating to the measured time via at least one communication interface of the time tracking device.
